(19) United States
(12) Patent Application Publication
(10) Pub. No.: US 2013/0052131 A1
(43) Pub. Date: Feb. 28, 2013

(54) NANOPARTICLES, METHODS OF MAKING NANOPARTICLES, AND METHODS OF USE

(71) Applicants: Zhen Cheng, Mountain View, CA (US); Kai Cheng, Mountain View, CA (US)

(72) Inventors: Zhen Cheng, Mountain View, CA (US); Kai Cheng, Mountain View, CA (US)

(73) Assignee: The Board of Trustees of the Leland Stanford Junior University, Palo Alto, CA (US)

(21) Appl. No.: 13/593,907

(22) Filed: Aug. 24, 2012

Related U.S. Application Data
(60) Provisional application No. 61/527,840, filed on Aug. 26, 2011.

(51) Int. Cl. A61K 49/00; G01N 29/14; C07K 2/00; C07H 21/00; C08B 37/00; C07J 51/00; C07F 19/00; A61K 38/02; A61K 31/715; A61K 39/00; A61K 31/282; A61K 31/56; A61K 31/555; A61P 35/00; A61P 37/04; A61K 49/22; C07H 23/00; A61K 51/02; A61K 31/7135; B22F 1/00; B32B 15/02; G01N 21/65; B82Y 5/00; B82Y 15/00

(52) U.S. Cl. 424/1.61; 435/7.23; 435/6.1; 435/7.1; 435/29; 530/300; 530/400; 536/23.1; 536/121; 552/504; 556/110; 544/225; 424/9.1; 514/1.1; 514/54; 424/193.1; 514/495; 514/169; 514/184; 514/186; 424/9.5; 514/23; 514/44 R; 420/591; 428/402; 977/906; 977/904; 977/774; 977/927

(57) ABSTRACT

Embodiments of the present disclosure, in one aspect, relate to a nanoparticle, methods of imaging a tumor, method of imaging a disease, method of treating a condition, disease, or related biological event, or the like.

### CROSS-REFERENCE TO RELATED APPLICATION

This application claims priority to U.S. provisional application entitled “NANOPARTICLES, METHODS OF MAKING NANOPARTICLES, AND METHODS OF USE,” having Ser. No. 61/527,840 filed on Aug. 26, 2011, which is entirely incorporated herein by reference.

### STATEMENT OF GOVERNMENTAL SUPPORT

This invention was made with government support under grant number U54CA119367, awarded by the National Institutes of Health (NIH) of the United States government. The government has certain rights in the invention.

### BACKGROUND

A variety of nanoparticles recently have been explored as novel agents for imaging and therapy of diseases such as cancer. Many engineered fluorescent or luminescent nanoparticles have been designed and applied as molecular probes for optical imaging in vitro and in preclinical animal models. However, their applications and potential clinical uses are hampered by the poor deep tissue imaging ability of the optical imaging modalities.

### SUMMARY

Embodiments of the present disclosure, in one aspect, relate to a nanoparticle, methods of imaging a tumor, method of imaging a disease, method of treating a condition, disease, or related biological event, or the like.

An embodiment of the present disclosure includes a composition, among others, having a nanoparticle having a platinum nanostructure and at least one gold nanostructure, wherein the platinum nanostructure and a gold nanostructure are directly attached to one another.

An embodiment of the present disclosure includes a method of imaging a tumor, among others, including: exposing a subject to an imaging device, wherein a nanoparticle is introduced to a subject, wherein the nanoparticle enters the tumor, wherein the nanoparticle has a platinum nanostructure and at least one gold nanostructure, wherein the platinum nanostructure and a gold nanostructure are directly attached to one another; and detecting the nanoparticles, wherein the location of the nanoparticles correlates to the location of the tumor.

An embodiment of the present disclosure includes a method of imaging a disease, among others, including: exposing a subject to an imaging device, wherein a nanoparticle is introduced to a subject, wherein the nanoparticle has a platinum nanostructure and at least one gold nanostructure, wherein the platinum nanostructure and a gold nanostructure are directly attached to one another, wherein the nanoparticle becomes disposed adjacent or within the diseased area; and detecting the nanoparticles, wherein the location of the nanoparticles correlates to the location of the disease.

An embodiment of the present disclosure includes a method of treating a condition, disease, or related biological event, among others, including: administering a pharmaceutically effective amount of a nanoparticle, wherein the nanoparticle has a platinum nanostructure and at least one gold nanostructure, wherein the platinum nanostructure and a gold nanostructure are directly attached to one another, wherein the nanoparticle has a targeting agent attached to the nanoparticle and wherein the targeting agent is selected from a polypeptide, an antigen, a nucleic acid, a polysaccharide, a sugar, a fatty acid, a steroid, a purine, a pyrimidine, a ligand, an aptamer, a small molecule, or a combination thereof, wherein the targeting agent has an affinity for a condition, disease, or related biological event or other chemical, biochemical, and/or biological events of the condition, disease, or biological event.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 illustrates a TEM of dumbbell Au—Pt, Au-dipods, Au-tripods, and Au-tetrapods.

FIG. 2 illustrates a HRTEM and STEM of Au-tripods.

FIG. 3 illustrates an optical absorption of various Au-multipods. The absorptions were normalized by sample weight.

FIG. 4 illustrates a photoacoustic maximum intensity projection images through an agarose phantom containing decreasing number of U87MG tumor cells exposed to RGD-Au-Tripods.

FIG. 5 illustrates a photoacoustic detection of tripods in living mice. The mice were injected subcutaneously with RGD-Au-Tripods at concentrations of 0.39 nM ˜12.5 nM.

FIG. 6 illustrates a RGD-Au-Tripod tumor targeting in living mice. Ultrasound (gray) and photoacoustic (lighter gray) images of coronal and sagittal slices though the tumor were shown in the columns at different time points.

FIG. 7 illustrates biodistribution data taken from various excised organs after injection of RGD-Au-Tripods with/without blocking dose of RGD at 72 hours post injection.

FIG. 8 illustrates the image analysis of Pt NPs. Representative TEM images of 4.9 nm truncated cubic (A1), 5.8 nm truncated cubic (B1), 6.5 nm cubic (C1) and 7.5 nm cubic (D1) Pt NPs. The Representative HRTEM images of corresponding Pt NPs were shown in A2-D2. The lattice fringes in the HRTEM images were corresponding to the (200) lattice planes.

FIG. 9 illustrates the particle size and distribution of various Pt NPs.

FIG. 10 illustrates the statistic number of particles with different Au-branch numbers. The results were obtained by counting 100 particles on TEM images.

FIG. 11 illustrates STEM-EELS of Au-tripods.

FIG. 12 illustrates the partial size and distribution of dumbbell Au—Pt, Au-dipods, Au-tripods, and Au-tetrapods.

FIG. 13 illustrates the hydrodynamic size of Au-Tripod-NH₂ (peak to left) and Au-Tripod-RGD (peak to right).

FIG. 14 illustrates the stability test of Au-Tripod-RGD.

FIG. 15 illustrates the specific targeting of RGD-Au-Tripods to U87MG cells. Reflection imaging of U87MG cells treated with RGD-Au-tripods for 4 hours.

FIG. 16 illustrates the internalization of RGD-Au-Tripods in U87MG cells. TEM images of cell sections were shown.

FIG. 17 illustrates the cellular uptake of RGD-Au-Tripods

FIG. 18 illustrates photoacoustic phantom images of Au-Tripods and Au-rods. Concentration of each particle was normalized by the sample weight.

FIG. 19 illustrates the photoacoustic signal from each inclusion was calculated suing 3D ROI. The error bars represents the standard error (n=3 mice).

FIG. 20 illustrates Scheme 1, which describes the synthesis of Pt seeds and branched Au nanostructures.

FIG. 21 illustrates Scheme 2, which describes the synthesis of water-soluble Au-Tripods and synthesis of RGD-Au-Tripods.

FIG. 22 illustrates Table 1, where the mean diameters and standard deviation of various Pt NPs are shown. The particle diameters were determined by TEM analysis and DLS.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of imaging, chemistry, material science, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

### DEFINITIONS

In describing and claiming the disclosed subject matter, the following terminology will be used in accordance with the definitions set forth below.

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art. As appropriate, procedures involving the use of commercially available kits and reagents are generally carried out in accordance with manufacturer defined protocols and/or parameters unless otherwise noted.

The term “detectable” refers to the ability to detect a signal over the background signal.

The term “photoacoustic imaging” describes signal generation caused by a light pulse, absorption, and expansion of an agent (particle), followed by acoustic detection, where the agent absorbs the light energy and converts it to thermal energy that generates the photoacoustic signal (also referred to as a “acoustic signal” that is derived from a particle that can generate a photoacoustic signal).

The term “acoustic detectable signal” is a signal derived from a particle that absorbs light and converts absorbed energy into thermal energy that causes generation of acoustic signal through a process of thermal expansion. The acoustic detectable signal is detectable and distinguishable from other background acoustic signals that are generated from the subject or sample. In other words, there is a measurable and statistically significant difference (e.g., a statistically significant difference is enough of a difference to distinguish among the acoustic detectable signal and the background, such as about 0.1%, 1%, 3%, 5%, 10%, 15%, 20%, 25%, 30%, or 40% or more difference between the acoustic detectable signal and the background) between acoustic detectable signal and the background. Standards and/or calibration curves can be used to determine the relative intensity of the acoustic detectable signal and/or the background.

The term “acoustic signal” refers to a sound wave produced by one of several processes, methods, interactions, or the like (including light absorption) that provides a signal that can then be detected and quantitated with regards to its frequency and/or amplitude. The acoustic signal can be generated from one or more particles of the present disclosure. In an embodiment, the acoustic signal may need to be sum of each of the individual photoacoustic signals. In an embodiment, the acoustic signal can be generated from a summation, an integration, or other mathematical process, formula, or algorithm, where the acoustic signal is from one or more probes. In an embodiment, the summation, the integration, or other mathematical process, formula, or algorithm can be used to generate the acoustic signal so that the acoustic signal can be distinguished from background noise and the like. It should be noted that signals other than the acoustic signal can be processed or obtained is a similar manner as that of the acoustic signal.

The acoustic signal or acoustic energy can be detected and quantified in real time using an appropriate detection system. One possible system is described in the following references: Journal of Biomedical Optics—March/April 2006—Volume 11, Issue 2, 024015, Optics Letters, Vol. 30, Issue 5, pp. 507-509, each of which are included herein by reference. In an embodiment, the acoustic energy detection system can includes a 5 MHz focused transducer (25.5 mm focal length, 4 MHz bandwidth, F number of 2.0, depth of focus of 6.5 mm, lateral resolution of 600 μm, and axial resolution of 380 μm. A3095-SU-F-24.5-MM-PTF, Panametrics), which can be used to acquire both pulse-echo and photoacoustic images. In addition, high resolution ultrasound images can also be simultaneously acquired using a 25 MHz focused transducer (27 mm focal length, 12 MHz bandwidth, F number of 4.2, depth of focus of 7.5 mm, lateral resolution of 250 μm, and axial resolution of 124 μm. V324-SU-25.5-MM, Panametrics). Other detection strategies including capacitive micromachined ultrasonic transducers (CMUT) arrays can also be used to detect the acoustic signal.

The term “optical detectable signal” is a signal derived from a particle that absorbs light and converts absorbed energy into optical energy of a different wavelength. The optical detectable signal is detectable and distinguishable from other background optical signals that are generated from the subject or sample. In other words, there is a measurable and statistically significant difference (e.g., a statistically significant difference is enough of a difference to distinguish among the optical detectable signal and the background, such as about 0.1%, 1%, 3%, 5%, 10%, 15%, 20%, 25%, 30%, or 40% or more difference between the optical detectable signal and the background) between optical detectable signal and the background. Standards and/or calibration curves can be used to determine the relative intensity of the optical detectable signal and/or the background.

The term “dispose” describes the permanent or temporary attachment of matter to a supporting material.

The term “illuminating” as used herein refers to the application of a light source, including near-infrared (NIR), visible light, including laser light capable of exciting dyes and nanoparticle cores of the embodiments of the probes herein disclosed.

The term “in vivo imaging” as used herein refers to methods or processes in which the structural, functional, or physiological state of a living being is examinable without the need for a life ending sacrifice.

The term “non-invasive in vivo imaging” as used herein refers to methods or processes in which the structural, functional, or physiological state of a being is examinable by remote physical probing without the need for breaching the physical integrity of the outer (skin) or inner (accessible orifices) surfaces of the body.

The term “sample” can refer to a tissue sample, cell sample, a fluid sample, and the like. The sample may be taken from a subject. The tissue sample can include brain, hair (including roots), buccal swabs, blood, saliva, semen, muscle, or from any internal organs, or cancer, precancerous, or tumor cells associated with any one of these. The fluid may be, but is not limited to, urine, blood, ascites, pleural fluid, spinal fluid, and the like. The body tissue can include, but is not limited to, brain, skin, muscle, endometrial, uterine, and cervical tissue or cancer, precancerous, or tumor cells associated with any one of these. In an embodiment, the body tissue is brain tissue or a brain tumor or cancer.

The term “administration” refers to introducing a probe of the present disclosure into a subject. One preferred route of administration of the compound is oral administration. Another preferred route is intravenous administration. However, any route of administration, such as topical, subcutaneous, peritoneal, intraarterial, inhalation, vaginal, rectal, nasal, introduction into the cerebrospinal fluid, or instillation into body compartments can be used.

As used herein, the term “host,” “subject,” or “patient,” includes humans and mammals (e.g., mice, rats, pigs, cats, dogs, and horses). Typical hosts to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For diagnostic or research applications, a wide variety of mammals will be suitable subjects, including rodents (e.g., mice, rats, hamsters), rabbits, primates, and swine such as inbred pigs and the like. The term “living host” refers to a host noted above or another organism that is alive. The term “living host” refers to the entire host or organism and not just a part excised (e.g., a liver or other organ) from the living host.

### GENERAL DISCUSSION

Embodiments of the present disclosure provide for compositions including nanoparticles, methods of using the nanoparticles, methods of making the nanoparticles, methods of imaging a condition, and the like. Embodiments of the present disclosure include nanoparticles that can be used to image, detect, study, monitor, evaluate, screen, and/or treat a sample or subject (e.g., whole-body or a portion thereof), in vivo and/or in vitro. Embodiment of the present disclosure can be advantageous in that the nanoparticles include two imaging modalities, photoacoustic and optical imaging modalities, in a single particle. In addition, the nanoparticles include noble metals that can serve as an attachment point for one or more types of agents. Furthermore, the size and morphology of the nanoparticle can be controlled and optimized for desired applications.

In an embodiment, the nanoparticle includes a platinum nanostructure and at least one gold nanostructure, where the platinum nanostructure and a gold nanostructure are directly attached to one another. In an embodiment, the growth of gold nanostructures on platinum nanostructures is directed by the lattice direction of platinum seeds during a seed-mediated process, eventually leading to a branched structure. The spatial distribution of gold branches on the platinum nanostructures is dependent on the size of platinum seeds.

In an embodiment, the diameter or the effective diameter (e.g., if the nanoparticle is rotated about a center of rotation (e.g., to form an imaginary sphere) and the outer most distance from the center of rotation reached (the edges of the imaginary sphere) defines the effective diameter) can be about 5 nm to 40 nm, about 5 nm to 25 nm, about 6 nm to 20 nm, or about 8 to 16. In an embodiment, the dimensions of the nanoparticle can be determined and measured using TEM.

In an embodiment, the nanoparticle includes a platinum nanostructure and one gold nanostructure, where the platinum nanostructure and the gold nanostructure are directly attached to one another to form a dumbbell shape. In an embodiment, the gold nanostructures are not attached directly to one another. In an embodiment, the diameter or the effective diameter of the nanoparticle can be about 8 to 14 nm. In an embodiment, the diameter of the platinum nanostructure can be about 2 to 4 nm or about 3 nm. In an embodiment, the diameter of the gold nanostructure can be about 3 to 5 nm or about 4 nm. An image of the dumbbell shape is shown in the Examples.

In an embodiment, the nanoparticle includes a platinum nanostructure and two gold nanostructures, where each of the two gold nanostructures is directly attached to the platinum nanostructure to form a dipod shape. In an embodiment, the gold nanostructures are not attached directly to one another. In an embodiment, the diameter or the effective diameter of the nanoparticle can be about 9 to 16 nm. In an embodiment, the diameter of the platinum nanostructure can be about 3 to 5 nm or about 4 nm. In an embodiment, the diameter of the gold nanostructure can be about 3 to 6 nm or about 4 to 5 nm. An image of the dipod shape is shown in the Examples.

In an embodiment, the nanoparticle includes a platinum nanostructure and three gold nanostructures, where each of the three gold nanostructures is directly attached to the platinum nanostructure to form a tripod shape. In an embodiment, the gold nanostructures are not attached directly to one another. In an embodiment, the diameter or the effective diameter of the nanoparticle can be about 15 to 21 nm. In an embodiment, the diameter of the platinum nanostructure can be about 5 to 7 nm or about 6 nm. In an embodiment, the diameter of the gold nanostructure can be about 5 to 7 nm or about 6 nm. An image of the tripod shape is shown in the Examples.

In an embodiment, the nanoparticle includes a platinum nanostructure and four gold nanostructures, where each of the four gold nanostructures is directly attached to the platinum nanostructure to form a tetrapod shape. In an embodiment, the gold nanostructures are not attached directly to one another. In an embodiment, the diameter or the effective diameter of the nanoparticle can be about 19 to 24 nm. In an embodiment, the diameter of the platinum nanostructure can be about 7 to 9 nm or about 8 nm. In an embodiment, the diameter of the gold nanostructure can be about 5 to 8 nm or about 6 to 7 nm. An image of the tetrapod shape is shown in the Examples.

In an embodiment, the nanoparticle can include one or more agents (e.g., a chemical or biological agent). In an embodiment, the agent can include a targeting agent, a drug, a therapeutic agent, a radiological agent, a chemological agent, a small molecule drug, a biological agent (e.g., peptides, proteins, antibodies, antigens, and the like) and combinations thereof. In an embodiment, each agent can be disposed indirectly or directly on the nanoparticle.

In an embodiment, the targeting agent has an affinity for a target in a subject or a tissue or fluid sample from a subject. In particular, the agent enables the particle to be used to image, detect, study, monitor, evaluate, screen, and/or treatment a disease, condition, or related biological event corresponding to the target, in vivo and/or in vitro.

In an embodiment, the targeting agent can function to cause the particle to interact with a molecule(s). In an embodiment, the targeting agent can have an affinity for a cell, a tissue, a protein, DNA, RNA, an antibody, an antigen, and the like, that may be associated with a condition, disease, or related biological event, of interest. In particular, the targeting agent can function to target specific DNA, RNA, and/or proteins of interest. The targeting agent can include, but is not limited to, polypeptides (e.g., proteins such as, but not limited to, antibodies (monoclonal or polyclonal)), antigens, nucleic acids (both monomeric and oligomeric), polysaccharides, sugars, fatty acids, steroids, purines, pyrimidines, ligands, aptamers, small molecules, ligands, or combinations thereof, that have an affinity for a condition, disease, or related biological event or other chemical, biochemical, and/or biological events of the condition, disease, or biological event. In an embodiment, the targeting agent can include: sequence-specific DNA oligonucleotides, locked nucleic acids (LNA), and peptide nucleic acids (PNA), antibodies, and small molecule protein receptors.

Embodiments of the present disclosure can be used to image, detect, study, monitor, and/or evaluate, a condition or disease such pre-cancerous tissue, cancer, or a tumor. In an embodiment, the method includes imaging pre-cancerous tissue, cancer, or a tumor. In particular, the particles of the present disclosure can be used to image a tumor since the particles can enter the tumor since they are relatively small. A method can include exposing a subject to an imaging device (e.g., photoacoustic detection system and/or optical energy detection system). The subject is given the particle prior to exposure and/or during exposure to the imaging device and after a period of time the particle enters the tumor. Subsequently, the particles are detected and the location of the particles can be determined. The location of the particles can be correlated with the location of the tumor and/or the presence of the tumor. Additional details are described in Examples.

Although the methods described above are directed to brain tumors, other tissue types can be substituted for the brain tumor. For example, pre-cancerous can cancer can be considered in the same way. In some embodiments, a targeting agent may need to be added to the probe for the probe to be disposed in the pre-cancerous or cancerous cells. As discussed in the Examples, no targeting agent is needed since the probe in the Example becomes disposed in the brain tumor without a targeting agent. Additional details are described in the Examples.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

### Brief Introduction

Photoacoustic imaging (PAI) provides functional and molecular information of tumors in real time and in high imaging contrasts and ultrasonic spatial resolution through endogenous and exogenous PAI contrasts. However, most of contrasts based on the nanostructures were currently suffering from unfavorable in vivo profiles. In this study, we developed novel branched gold nanostructures, Au-tripods, with small particle size (<20 nm) for enhanced photoacoustic response through stringently controlled morphology. The Au-tripods display well-defined absorption peaks in visible and near-infrared (NIR) region. The PAI signals were linearly correlated with their concentrations after subcutaneous injection. We conjugated them with cyclic Arg-Gly-Asp (RGD) peptides to molecularly target the αᵥβ₃ integrins. The in vivo biodistribution of Au-tripods was evaluated by ⁶⁴Cu radiolabeling and imaging their localization over time using micropositron emission tomography (PET). Intravenous administration of RGD conjugated Au-tripods to U87MG bearing mice showed remarkably higher contrast in tumors than the blocking ones even in the subnanomolar concentrations. The results correlated well with the corresponding PET images. Our study suggests that highly selective and sensitive detection of cancer cells in a living subject is possible using molecular specific Au-tripods as PAI contrast agents.

### Introduction

A variety of nanoparticles (NPs) recently have been explored as novel agents for imaging and therapy of cancers. Many engineered fluorescent or luminescent NPs have been designed and applied as molecular probes for optical imaging in vitro and in preclinical animal models. However, their applications and potential clinical uses are hampered by the poor deep tissue imaging ability of the optical imaging modalities. Photoacoustic imaging (PAI) is a newly emerged molecular imaging technique and has attracted significant research interests recently. PAI takes advantage of both optical and acoustic imaging and overcomes the depth limitation and can recognize small lesions at depths of up to 5 cm. It is a highly sensitive, non-invasive molecular imaging technique in which light enters the body to produce heat and sound exits. It can provide functional and molecular information of tumors in real time through endogenous and exogenous PAI contrast agents. It becomes a highly attractive molecular imaging technique and holds great promise for clinical translation. Therefore development of NPs suitable for PAI applications is very important and is expected to have high impact in cancer research and management.

The amplitude of photoacoustic signal and contrasts are dependent on many factors such as optical absorption, optical-to-acoustic conversion efficiency, and heat transfer of the target medium.¹¹ Various light-absorbing NPs, such as Au-based nanostructures, carbon nanotubes, have been studied as PAI probes.¹²⁻²⁵ Although carbon nanotubes have shown promising properties as contrast agents for PAI, their imaging sensitivity is relatively low because of the low NIR light absorption coefficient. Moreover, their toxicity remains a concern.²⁵ The Au nanostructures such as Au-nanorods, nanocages and nanoshells exhibit superior optical properties, biocompatibility and safety, and thus they have been actively investigated in the fields of optical imaging and PAI. When Au nanostructures interact with incident light, they give rise to a strong extinction peak in the visible and NIR regions because of the localized surface plasmon resonance. Such extinction peak comprised of two components, scattering and absorption, which can be tuned by simply controlling the size, shape and other physical dimensions of the nanostructures. Among various optical Au—NPs, ones with relatively dominant absorption over scattering are highly desirable for PAI. For example, Au-nanoshells with dielectric cores show strong optical resonance while relative weak optical absorption, and therefore they are not ideal candidates for PAI.^{20,26,27} To the contrast, Au-nanorods and nanocages exhibit non-spherical symmetry and their optical absorption cross sections (σₐ) in the NIR region could be maximized by manipulating their aspect ratios and thus are more suitable for PAI.¹¹

Although Au-nanorods and nanocages are good PAI enhancers, they generally have relatively large particle sizes (˜50 nm) and unfavorable in vivo profiles.^{11,15,18,25} In order to optimize the photoacoustic agents to achieve high imaging sensitivity and desired in vivo tumor targeting properties, novel Au—NPs with different shapes, sizes and optical properties should be explored. Previously many Au-nanostructures with complex shapes and irregular geometries such as branched NPs, nanostars, sea urchin-like NPs have been pursued.^{21,24,28-32} Unfortunately none of them have been evaluated for their applications in biomedical research. In this paper, we have synthesized novel Au-based nano-multipods including dipods, tripods and tetrapods with small size (<20 nm) and stringently controlled morphology. Among them, Au tripods displays small size, well-defined absorption peaks in visible and NIR region. Varying the particle shapes of these Au-tripods allows us to easily control the relative contributions of scattering and absorption at a given wavelength of interest, thus to control their PAI sensitivity as well. Moreover, Au-Tripods are known to be biologically inert and can be readily coated with water soluble organic layers and modified with biomolecules, which facilitates their in vivo use. To proof of concept, we prepared the conjugation of RGD peptides to pegylated Au-Tripods and focused on their use as photoacoustic imaging agents to image αᵥβ₃ integrins, which are associated with tumor angiogenesis. We further validated that RGD-Au-Tripods showed superior stability in serum and there were not significant cytotoxity with RGD-Au-Tripods. The in vivo biodistribution of Au-Tripods is evaluated by radiolabeling them with ⁶⁴Cu and imaging their localization and accumulation over time using PET and gamma counting followed by both transmission electron microscopy (TEM) and inductively coupled plasma mass spectrometry (ICP-MS) of removed tissues or organs for confirmation of their localization. The photoacoustic contrast after intravenous administration of RGD-Au-tripods to U87MG bearing tumor mice were compared with that of blocking ones.

### Results

### Synthesis and Characterization of Au-Tripods.

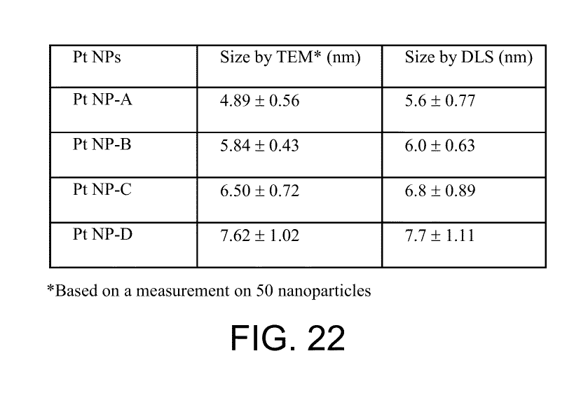

Au-Tripods were synthesized through a seed-mediated process (Scheme 15). When cubic or polyhedral platinum (Pt) NPs were used as seeds, Au precursor HAuCl₄ could be reduced by oleylamine in 1-octadecene and epitaxially grown on Pt seeds. Because the size and shape of Pt NPs are critical to this seed-mediated process, it is very important to first obtain monodisperse Pt NPs with desired shapes. Sun and coworkers reported a facile route to prepare monodisperse Pt NPs with tunable size from 3 nm to 7 nm. The shape and size of resultant Pt NPs are strongly dependent on reaction temperature at which a trace amount of iron pentacarbonyl (Fe(CO)₅) was injected. The trace Fe(CO)₅ was believed to facilitate fast nucleation and improve homogeneous growth of platinum, ending up with monodisperse Pt NPs. However, we found that it was hard to control their sizes and shapes during the synthesis of large Pt NPs (bigger than 6 nm). At the low reaction temperature, the unexpected low-nucleation during the synthesis of large NPs often results in poor, inconsistent, and non-reproducible quality of Pt NPs. In order to improve the quality, we tried to use small Pt NPs with high quality as seeds to grow the large Pt NPs (Scheme 1S). Typically, the 4.9 nm Pt NPs (Pt NP-A) were injected into the reaction mixture containing Pt precursor, followed by injection of trace Fe(CO)₅. Finally, truncated cubic Pt NPs became large cubic ones (6.5 nm, Pt NP—C) with same quality (FIG. 8). The large Pt NPs (Pt NP-D) with size more than 7.5 nm can also be obtained when 6 nm Pt NPs were used as seeds. All of the cubic Pt NPs have a very narrow size and shape distribution (FIG. 22 and Table 1). The lattice fringes of all four Pt NPs shown in FIG. 8 are c.a. 0.195 nm and related to (111) planes of Pt in fcc phase.

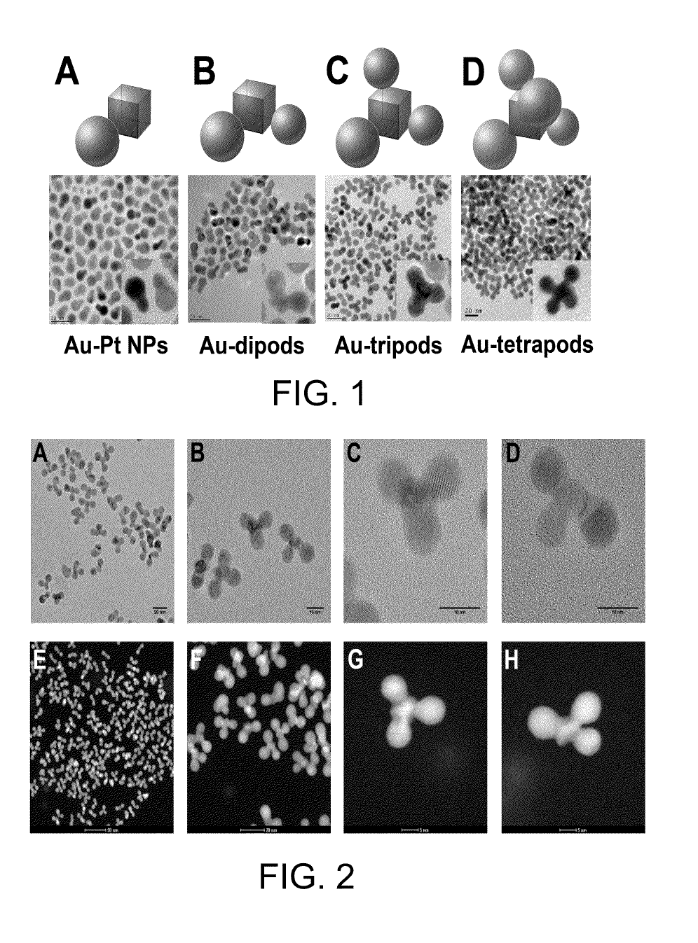

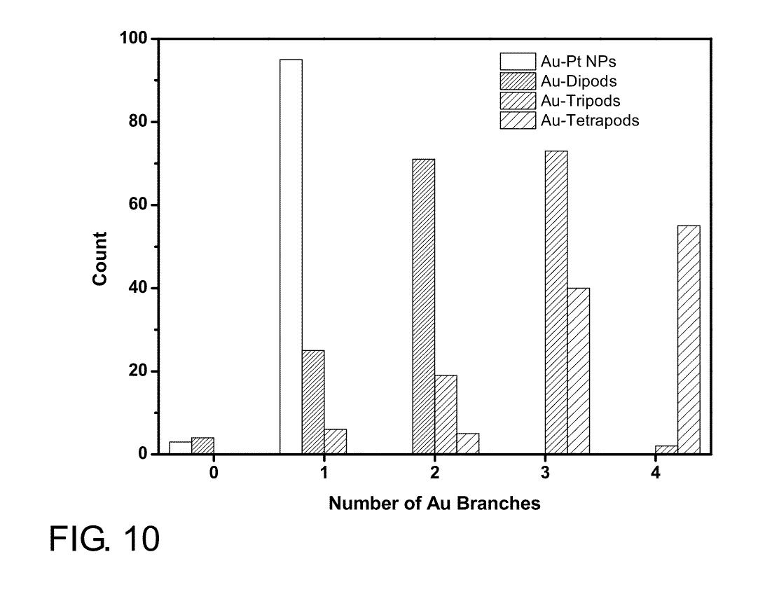

The use of Pt NPs with stringently controlled sizes and shapes as seeds results in various Au hetero-structures with controlled shapes, such as dumbbell-like Au—Pt NPs, Au-dipods, Au-tripods, or Au-tetrapods (FIG. 8 and FIG. 1). As Sun and coworkers described previously, the larger Pt NPs end up with more complex nanostructures. Typically, the epitaxial growth of Au on 4.9 nm Pt seeds produced dumbbell-like Au—Pt NPs. Use of 6 nm Pt NPs as seeds resulted in Au-Dipods. Au-Tetrapods could be obtained when 7.5 nm Pt NPs were used as seeds. Interestingly, we found that, unlike 7.5 nm Pt NPs resulting in a mixture of tripods and tetrapods, 6.5 nm Pt seeds almost exclusively led to formation of tripods with a narrow distribution of size and shape (FIG. 1C). The statistic analysis suggested that majority (more that 70%) of resultant nanoparticles were tripods (FIG. 10).

FIG. 1 illustrates a TEM of dumbbell Au—Pt, Au-dipods, Au-tripods, and Au-tetrapods.

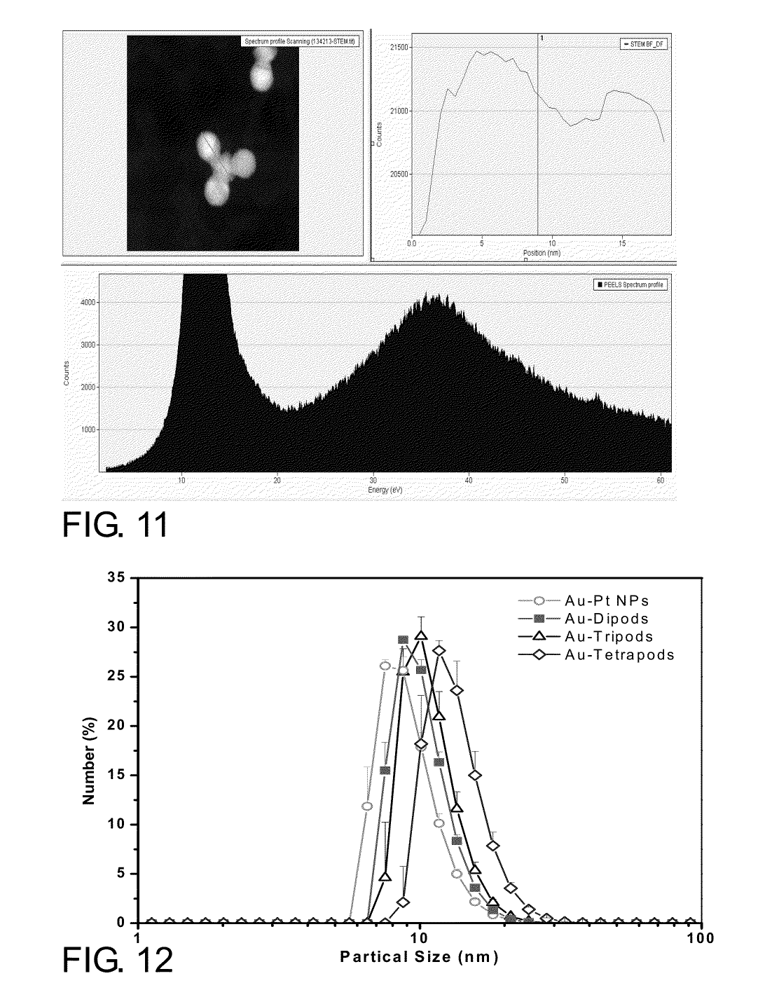

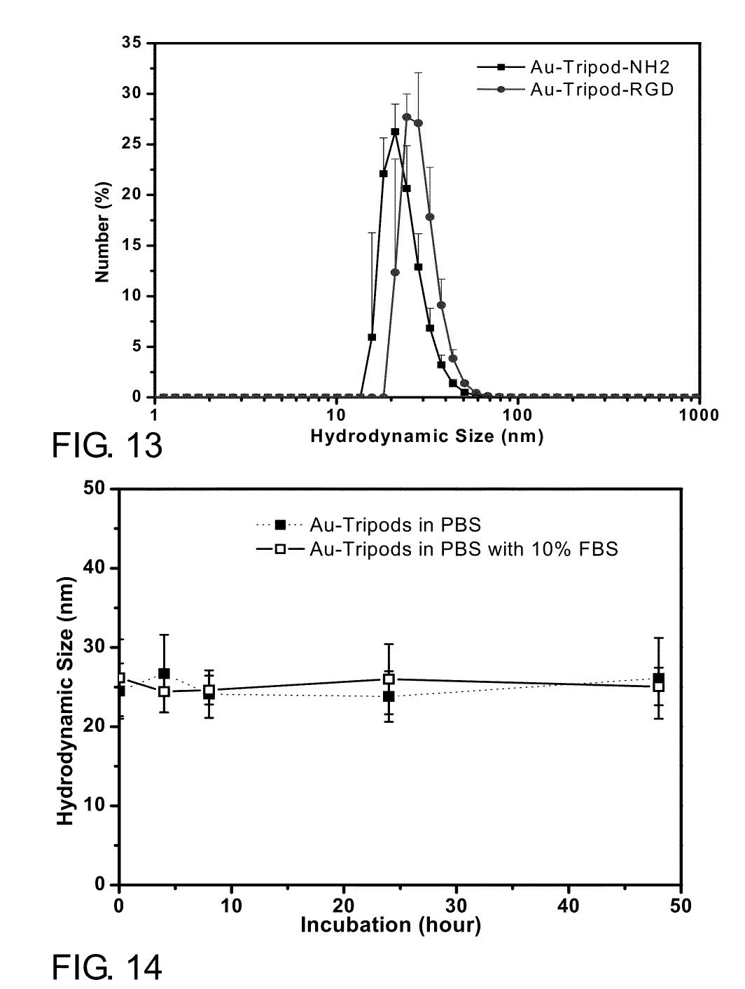

Unlike the other heterogeneous nucleation and growth, where different shapes could be achieved by tuning the seed-to-precursor ratio and controlling the heating profile, the growth of Au-multipods on Pt NPs is directed by the lattice direction of Pt seeds. The growth along the preferential lattice directions (i.e. (111)) is faster than that along other directions, leading to branched structures (multi-pods). The representative HRTEM and STEM images of Au-Tripods showed that well crystallized interfaces had been formed between the Pt seeds and grown Au branches because of their very similar lattice constants in an fcc phase. The lattice fringes between Pt seeds and Au braches shown in FIG. 2 are c.a. 0.23 nm and related to (111) planes of either Pt or Au in the fcc phase. STEM images clearly showed that each Au branch epitaxially grew on one of the corners of cubic Pt seeds. The Au branches and Pt seeds could be distinguished by STEM-EELS (FIG. 11). It further confirmed that the epitaxial growth of Au on Pt seeds preferentially happened on Pt (111) planes.

FIG. 2 illustrates HRTEM and STEM of Au-tripods.

We performed the DLS and statistic analysis based on TEM images for measuring the sizes of various Au multipods. It is worth noticing that the size of Au-tripods obtained by DLS is smaller than that measured by TEM analysis (FIG. 12). In the DLS, the diameter of a nonspherical particle is the diameter of a sphere that has the same translational diffusion speed as the particle. However, TEM images show the 2D views of the particles. The size of particles can only be measured from the top-view. The thickness of the Au-tripods is often related to the size of one of the Au-branch or Pt seeds.

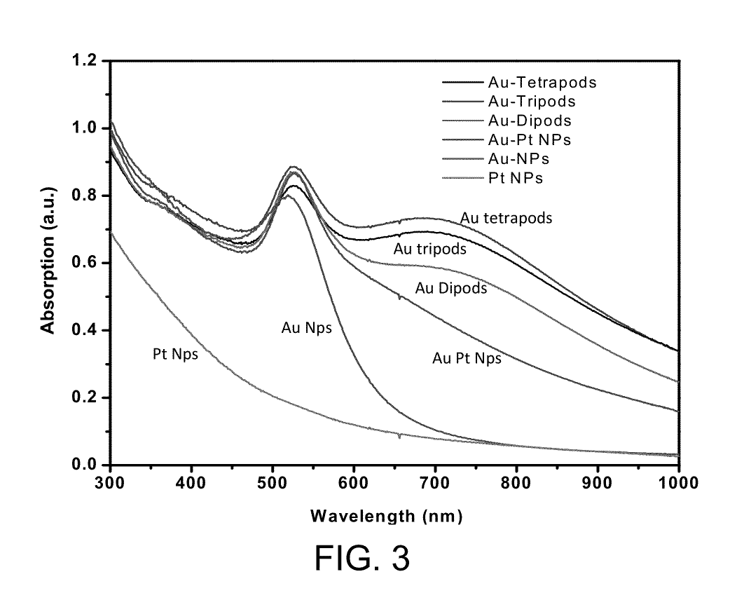

FIG. 3. Optical absorption of various Au-multipods. The absorptions were normalized by sample weight.

The PAI requires contrast agents with relatively large absorption cross section. A conventional UV-vis-NIR spectrometer could measure the extinction spectra of nanostructures, which are comprised of two components: scattering and absorption, eventually providing the extinction cross-section (σₑ, σₑ=σₐ+σₛ, where σₐ, is absorption cross section and σₛ is scattering cross section). As seen in FIG. 3F, the Au-tripods have a strong extinction peak with a broad shoulder in both visible and NIR regions. After deconvolution of the broad extinction peak from 400 to 1000 nm, two plasmon resonances at 540 nm and 700 nm can be distinguished. The 540 nm feature corresponded to a quadrupole resonance out of the plane of the gold tripods, and the 700 nm feature was attributed to a dipole resonance in the plane of the Au-tripods.²⁴ With varying the geometries and shapes, we expect the Au-tripods to have a strong optical absorption in the NIR region. The correlation between the morphology and optical spectra of Au-tripods is critical to design desired contrast enhancers for PAI and suggests that stringently controlled shapes could result in strong, discrete optical absorption peaks in visible-NIR regions.

### Surface Modification and In Vitro Targeting of Au-Tripods.

A surface modification step is necessary to provide the NPs water-soluble, biocompatible and functionalizable. The as-prepared Au-Tripods were typically coated with a hydrophobic hydrocarbon layer (oleylamine). To avoid undesired aggregation in physiological medium and provide an active functional group capable of conjugating with biological macromolecules, we developed a strategy based on the ligand exchange to anchor bifunctional polyethylene glycol to the Au surface via a thiol-gold interaction. As illustrated in Scheme 2S, the dithiol ligand, lipoic acid, was first converted to a succinimide ester which is capable of conjugating with one of primary amine groups of polyethylene glycol 3000. Lipoic acid terminated PEG-3000 refers to the approach of the ligand exchange that enables the phase transfer of the Au-tripods from organic solvents to aqueous solution and provides a steric barrier to prevent NPs agglomeration. Moreover, the PEG-3000-NH2 also offers different chemical functional groups (i.e. maleimide) and facilitate subsequent immobilization of various biological molecules via bioconjugation chemistry (Scheme 2S). Thus the RGDyC can site-specifically conjugate with the maleimide-terminated NPs in an oriented and homogeneous fashion. In order to non-invasively track RGD-Au-Tripods in vivo by PET, radiometal chelator, 1,4,7,10-tetra-azacyclododecane-1,4,7,10-tetraacetic acid (DOTA) will also be conjugated with RGD-Au-Tripods in a well defined manner for ⁶⁴Cu radiolabeling (Scheme 2S). Conjugation of the metal chelator with the amine group presented on NPs coating did not diminish the bioactivity of the RGD.

The hydrodynamic size of Au-Tripods slightly increased after conjugated with RGD peptide (FIG. 13). In the contrast, the zeta potentials dramatically changed from positive charge (+15 mV) to negative charge (−18 mV). RGD-Au-Tripods show excellent stability at the physiological condition. There were not significant size changes or aggregation even in the presence of the serum after 48 hours at 37° C.

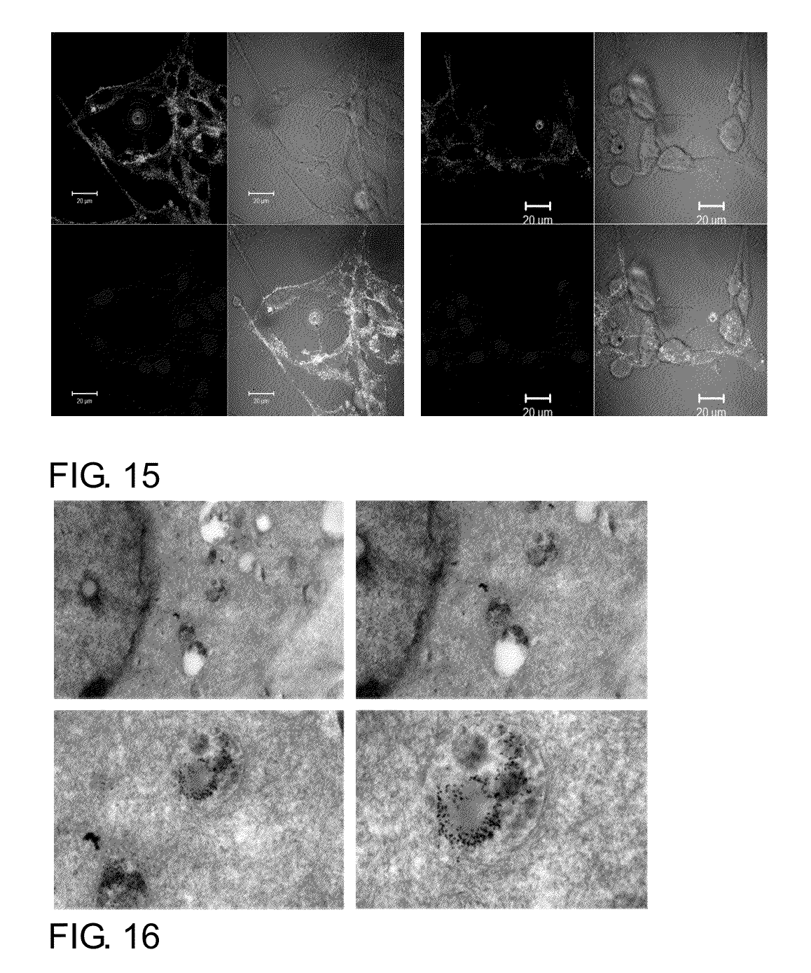

The receptor binding ability and specificity of RGD-Au-Tripods for the αᵥβ₃ integrins were evaluated in U87MG cells. As seen in FIG. 15, U87MG cells were identified by a DAPI nucleic acid dye. The cellular uptake and internalization of RGD-Au-Tripods were visualized as golden dots in the cells. A significant amount of golden RGD-Au-Tripods were associated with cells, compared to that of blocking ones.

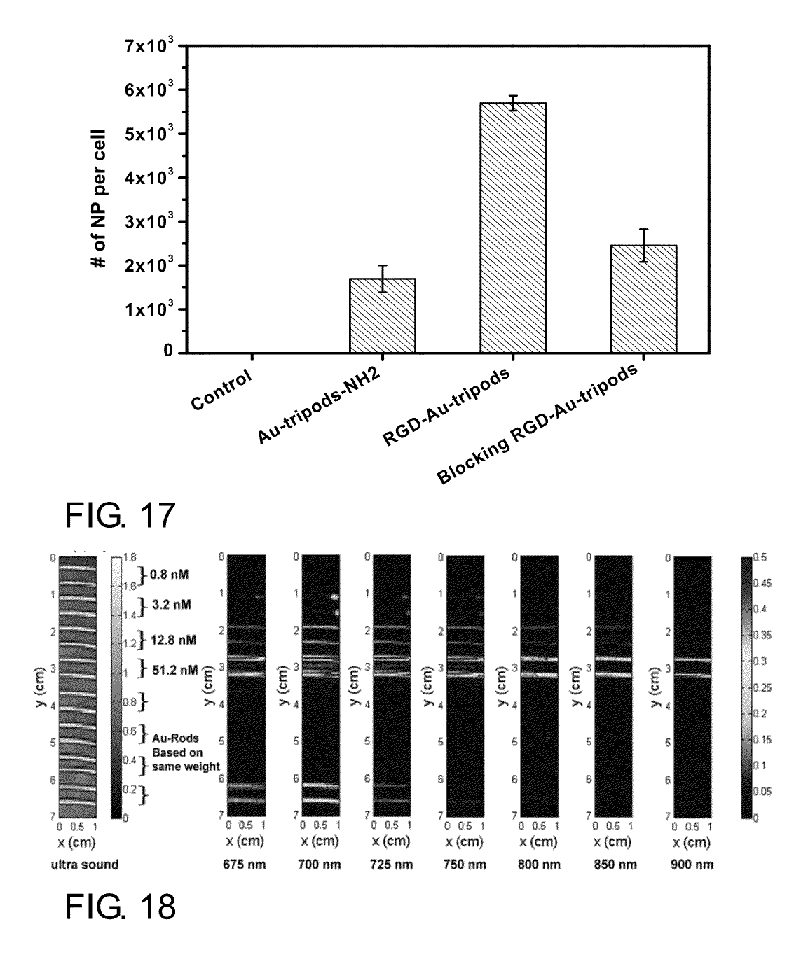

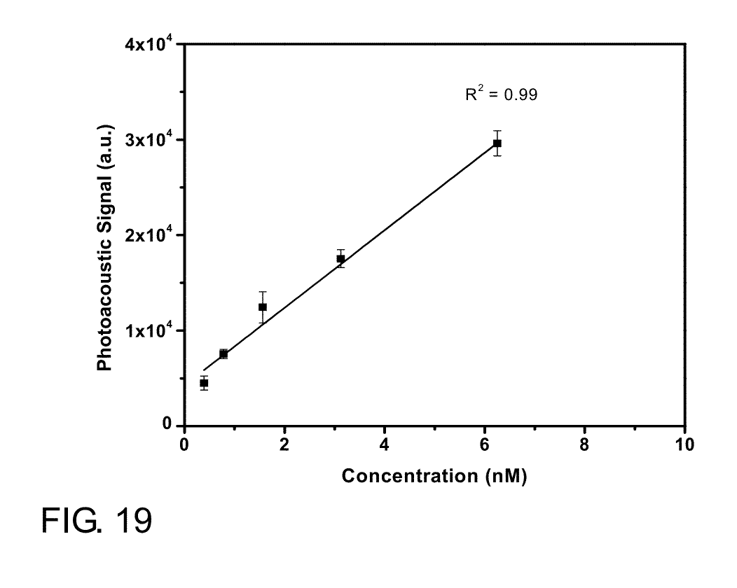

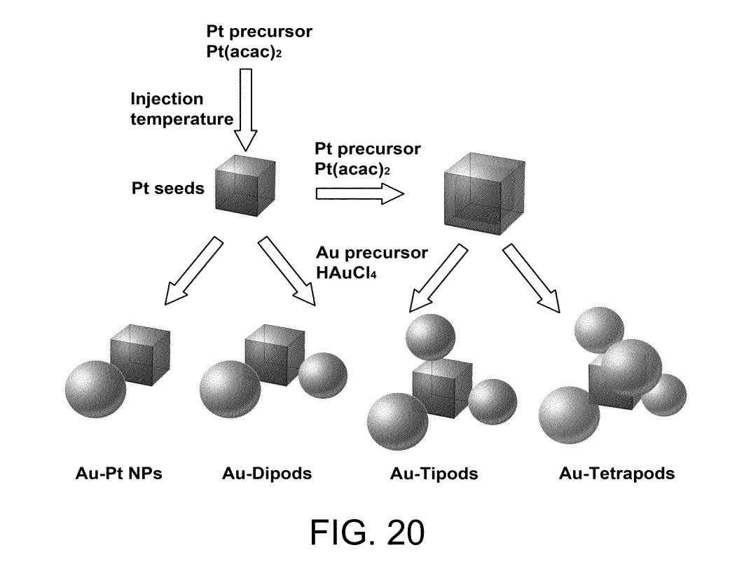

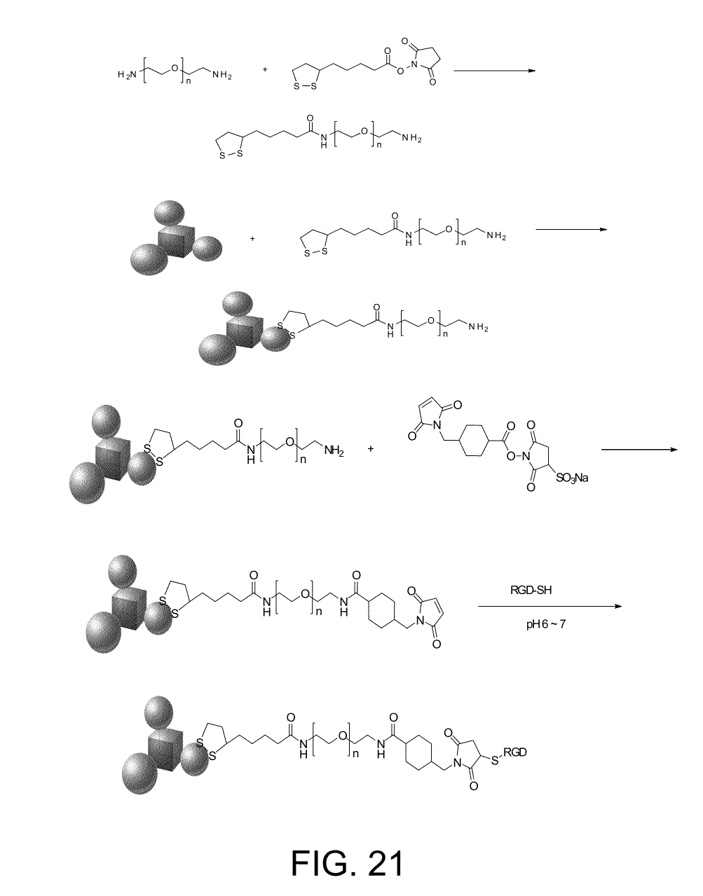

The receptor binding ability and specificity of RGD-Au-Tripods for the αᵥβ₃ integrins were evaluated in U87MG cells. As seen in FIG. 15, the cellular uptake and/or internalization of RGD-Au-Tripods were visualized as golden dots in the reflection images (FIG. 15). A significant amount of RGD-Au-Tripods were associated with cells, compared to that of blocking ones. The cellular uptake of RGD-Au-Tripods was significantly reduced by pre-treatment of the cells with excess RGD peptide. ICP-MS confirmed that the Au concentration in U87MG cells treated with RGD-Au-Tripods was 5700 particle/cell, and only 1700 particle/cell when cells were treated with Au-Tripods (FIG. 17). Pre-treatment of U87MG cells with RGD peptide reduced Au concentration to levels for non-targeting Au-Tripods. These results suggested that RGD coupled to Au-Tripods facilitated specific targeting of Au-Tripods to the αᵥβ₃ integrins.

### Sensitivity of Au-Tripods for Photoacoustic Molecular Imaging.

The absorption cross section σₐ of Au-tripods can be experimentally measured using photoacoustic sensing. The in-house photoacoustic system was setup and illustrated in our previous work.^{16,59} Polyethylene capillaries could be filled with various NPs and embedded in the 0.75% agar gel. A tunable pulsed laser with a repetition rate of 10 Hz and a pulse width of 5 ns was focused on to the phantom sample. The average laser light intensity at the range of 675 to 1000 nm wavelengths was measured to be ˜2 mJ/cm² on the sample surface. We constructed an agarose phantom with inclusions of Au-tripods at increasing concentrations from 0.8 nM to 51.2 nM (normalized by particle number). The photoacoustic signal produced by Au-tripods was observed to be linearly dependent on the concentrations (FIG. 18). Compared with regular Au-Rods with similar absorption peak at the same concentration (normalized by sample weight),⁶⁰ Au-tripods produced much higher photoacoustic signal in the range of 675 and 900 nm, leading to subnanomolar sensitivities.

To test the minimum detection level of RGD-Au-Tripods to the U87MG cells, we incubated these cells with particle solution for 4 hours. After incubation, the cells were washed three times with cold saline to remove unbound particles and placed in a clear agarose phantom at increasing concentrations from 11.5 K to 368 K cells per well (n=2 samples per group) and imaged with the photoacoustic system. Quantitative analysis of the photoacoustic signal from the phantom suggested that cells exposed to RGD-Au-Tripods were detected even at relatively low concentration (as low as 2.3×10⁴).

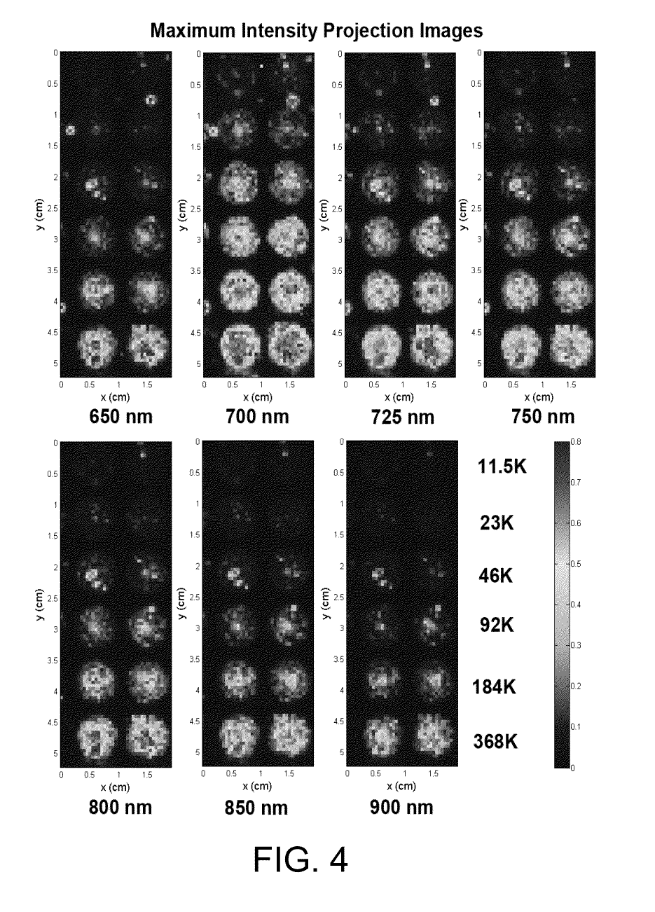

FIG. 4 illustrates photoacoustic maximum intensity projection images through an agarose phantom containing decreasing number of U87MG tumor cells exposed to RGD-Au-Tripods.

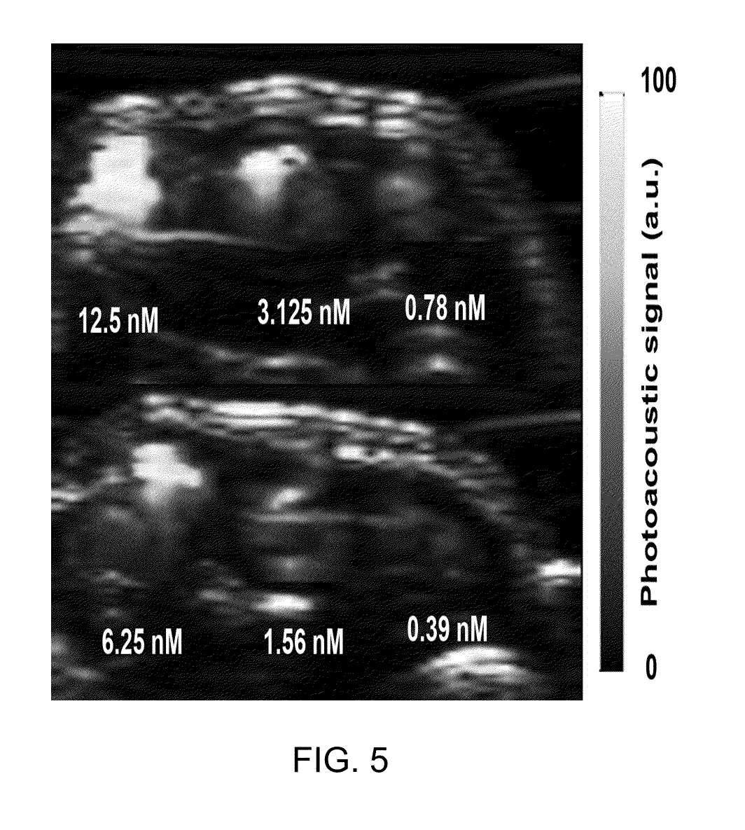

We then tested the sensitivity of RGD-Au-Tripods in living subjects by subcutaneously injecting the low side-back of mice (n=3) with 50 ul of RGD-Au-Tripods mixed with matrigel at increasing concentrations of 390 μM to 12.5 nM. Upon injection, the solidified matrigel fixed the NPs in the inclusions and three-dimensional ultrasound and photoacustic images of the inclusions were acquired (FIG. 5). The images represent ultrasound (gray) and photoacoustic (green) vertical slices through the subcutaneous injections. The photoacoustic signal from each inclusion was quantified using a 3D region of interest (ROI) covering the inclusion volume. We found that photoacoustic signal linearly correlated with the concentration of RGD-Au-Tripods (FIG. 18).

FIG. 5 illustrates photoacoustic detection of tripods in living mice. The mice were injected subcutaneously with RGD-Au-Tripods at concentrations of 0.39 nM˜12.5 nM.

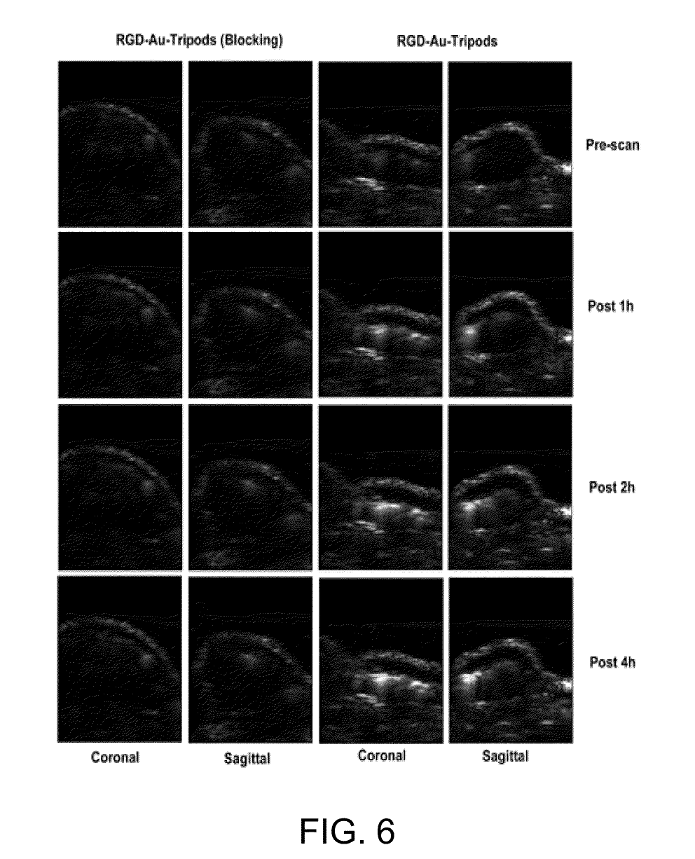

Finally, we studied the targeting ability of RGD-Au-Tripods in living mice. Mice bearing U87MG tumor xenografts were injected through the tail vein with 100 μl of either RGD-Au-Tripods with blocking dose of RGD (0.3 mg) or RGD-Au-Tripods alone at a concentration of 100 nM. We acquired 3D photoacoustic and ultrasound images of the entire tumor area before and up to 4 hour after the injection. Mice injected with RGD-Au-Tripods showed significantly higher photoacoustic signal in the tumor compared with the control group injected with both RGD-Au-Tripods and a blocking dose of RGD (FIG. 6). The ultrasound images were used for visualizing the boundaries of the tumor. The background signal of tumor before the injection is primarily due to the tumor's blood content. The significant increase in the photoacoustic signal after the injection is due to the Au-Tripods. Quantitative measurement of the photoacoustic signal from a 3D ROI around the tumor showed that the photoacoustic signal in the tumor was significantly higher in mice injected with both RGD-Au-Tripods and RGD. In instance, the mice after the injection of RGD-Au-Tripods showed relatively high photoacoustic signal in the tumor after 2 hours.

FIG. 6 illustrates RGD-Au-Tripod tumor targeting in living mice. Ultrasound (gray) and photoacoustic (light gray) images of coronal and sagittal slices though the tumor were shown in the columns at different time points.

### MicroPET Imaging and Biodistribution Data.

To image and track RGD-Au-Tripods in vivo by microPET, the mice bearing U87MG tumor (n=6 for each group) were injected with ⁶⁴Cu labeled DOTA-RGD-Au-Tripods with or without blocking dose of RGD through the tail vein, and the mice were imaged at different time post injection (0.5, 1, 2, 4, 24, 48, and 72 hours). Static microPET images acquired at various time points post injection over 72 hours revealed significant uptake of RGD-Au-Tripods within the liver and spleen.

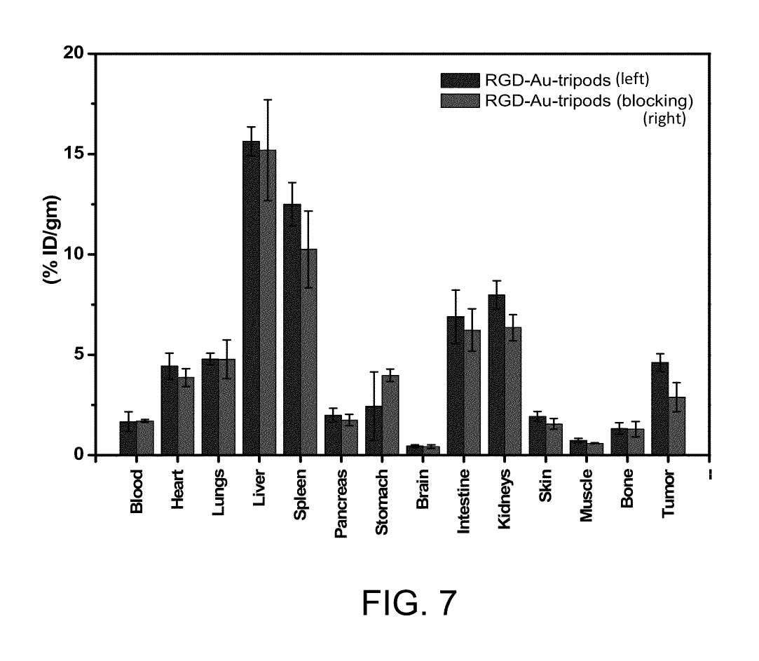

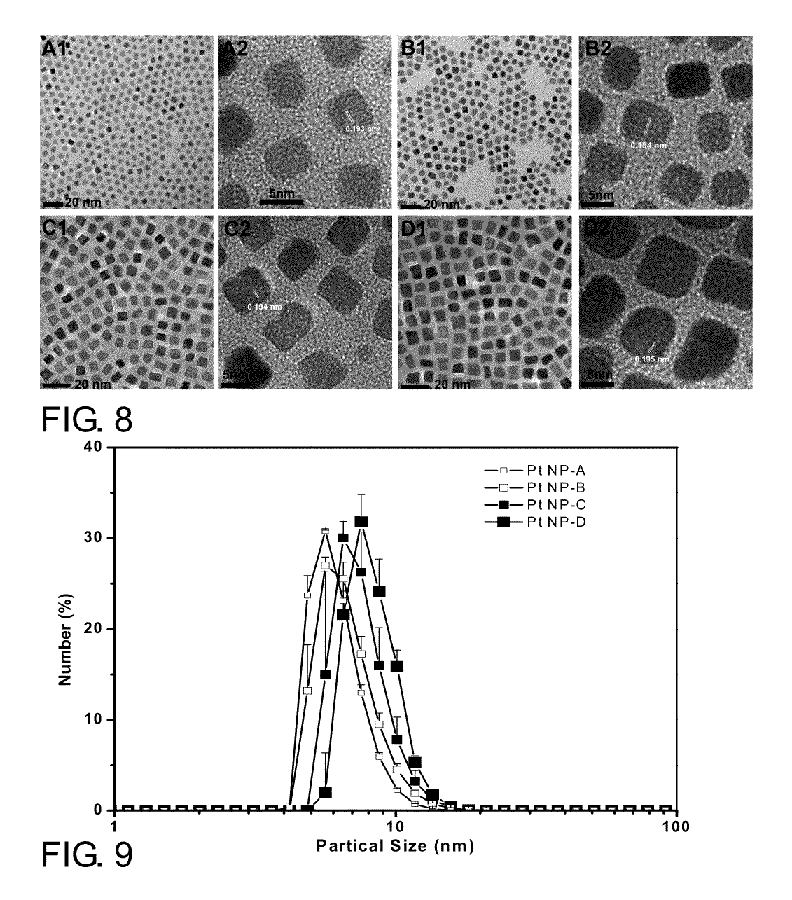

FIG. 7 illustrates the biodistribution data taken from various excised organs after injection of RGD-Au-Tripods with/without blocking dose of RGD at 72 hours post injection.

### Results and Discussion

Here, we designed a novel Au-based anisotropic nanostructures with stringently controlled shapes could exhibit superior optical properties and their optical absorption wavelength could be tuned in the visible and near infrared (NIR) region, therefore they could serve as promising photoacoustic contrast agents. We prepared ultra-small Au-tripod NPs (<20 nm) with strong NIR optical absorption by varying their geometries and shapes. In a phantom study and subcutaneous experiment, the photoacoustic spectra suggested that 700 nm is the preferable wavelength for in vivo experiment and the normalized intensity of the PAI signal of Au-tripods is as high as that of carbon nanotubes and slightly higher than that of tuned gold nanorods at the same wavelength. After conjugated with cyclic Arg-Gly-Asp (RGD) peptides, the targeting ability of c(RGDyK)-Au-tripods to integrin αvβ3 in vivo was evaluated with mice bearing U87MG tumors. After intravenous injection, these NPs were found to selectively concentrate on U87MG tumor cells. Compared to gold nanorods, Au-tripods with 20 nm of hydrodynamic size have less the reticuloendothelial system (RES) uptake and prolonged circulation time, by which they have enough time to reach the target sites. Radio labels (i.e. 64Cu) were introduced for tracking and imaging of Au-tripods in real time in vivo. PET provided three-dimensional distribution information of radiolabeled NPs in the live mice in real time. These quantitative kinetic results were further verified ex vivo using PAI and elemental analysis. In a short, the novel gold nanotripods could be an effective alternative to other existing NPs for non-invasive targeted molecular imaging in vivo. They could also be used as a delivery vehicle for potential treatment applications.

References for Example 1, each of which is incorporated herein by reference
1. Hong W K, Bromer R. Current Concepts—Chemotherapy in Head and Neck-Cancer. New Engl J Med. 1983; 308(2):75-9.
2. Vokes E E, Weichselbaum R R, Lippman S M, Hong W K. Medical Progress—Head and Neck-Cancer. New Engl J Med. 1993; 328(3):184-94.
3. Seiwert T Y, Cohen E E W. State-of-the-art management of locally advanced head and neck cancer. British Journal of Cancer. 2005; 92(8):1341-8.
4. Lee J J, Hong W K, Hittelman W N, Mao L, Lotan R, Shin D M, et al. Predicting cancer development in oral leukoplakia: Ten years of translational research. Clin Cancer Res. 2000; 6(5):1702-10.
5. Weigum S E, Floriano P N, Redding S W, Yeh C K, Westbrook S D, McGuff H S, et al. Nano-Bio-Chip Sensor Platform for Examination of Oral Exfoliative Cytology. Cancer Prev Res. 2010; 3(4):518-28.
6. Parkin D M, Bray F, Ferlay J, Pisani P. Global cancer statistics, 2002. Ca-Cancer J Clin. 2005; 55(2):74-108.
7. Bernier J, Bentzen S M, Vermorken J B. Molecular therapy in head and neck oncology. Nat Rev Clin Oncol. 2009; 6(5):266-77.
8. Spafford M F, Koch W M, Reed A L, Califano J A, Xu L H, Eisenberger C F, et al. Detection of head and neck squamous cell carcinoma among exfoliated oral mucosal cells by microsatellite analysis. Clin Cancer Res. 2001; 7(3):607-12.
9. vanderWaal I, Schepman K P, vanderMeij E H, Smeele L E. Oral leukoplakia: a clinicopathological review. Oral Oncol. 1997; 33(5):291-301.
10. Yang X, Stein E W, Ashkenazi S, Wang L V. Nanoparticles for photoacoustic imaging. Wiley Interdiscip Rev Nanomed Nanobiotechnol. 2009; 1(4):360-8.
11. Kim C, Favazza C, Wang L V. In Vivo Photoacoustic Tomography of Chemicals: High-Resolution Functional and Molecular Optical Imaging at New Depths. Chemical Reviews. 2010; 110(5):2756-82.
12. Kim C, Cho E C, Chen J, Song K H, Au L, Favazza C, et al. In Vivo Molecular Photoacoustic Tomography of Melanomas Targeted by Bioconjugated Gold Nanocages. ACS Nano. 2010; 4(8):4559-64.
13. Agarwal A, Huang S W, O'Donnell M, Day K C, Day M, Kotov N, et al. Targeted gold nanorod contrast agent for prostate cancer detection by photoacoustic imaging. Journal of Applied Physics. 2007; 102(6):064701.
14. Mallidi S, Larson T, Tam J, Joshi P P, Karpiouk A, Sokolov K, et al. Multiwavelength photoacoustic imaging and plasmon resonance coupling of gold nanoparticles for selective detection of cancer. Nano Lett. 2009; 9(8):2825-31. PMCID: 2898720.
15. Song K H, Kim C, Cobley C M, Xia Y, Wang L V. Near-Infrared Gold Nanocages as a New Class of Tracers for Photoacoustic Sentinel Lymph Node Mapping on a Rat Model. Nano Lett. 2008; 9(1):183-8.
16. De La Zerda A, Zavaleta C, Keren S, Vaithilingam S, Bodapati S, Liu Z, et al. Carbon nanotubes as photoacoustic molecular imaging agents in living mice. Nat Nanotechnol. 2008; 3(9):557-62.
17. Galanzha E I, Shashkov E V, Kelly T, Kim J W, Yang L L, Zharov V P. In vivo magnetic enrichment and multiplex photoacoustic detection of circulating tumour cells. Nat Nanotechnol. 2009; 4(12):855-60.
18. Kim J W, Galanzha E I, Shashkov E V, Moon H M, Zharov V P. Golden carbon nanotubes as multimodal photoacoustic and photothermal high-contrast molecular agents. Nat Nanotechnol. 2009; 4(10):688-94.
19. Jin Y D, Jia C X, Huang S W, O'Donnell M, Gao X H. Multifunctional nanoparticles as coupled contrast agents. Nat Commun. 2010; 1:-.
20. Nehl C L, Hafner J H. Shape-dependent plasmon resonances of gold nanoparticles. J Mater Chem. 2008; 18(21):2415-9.
21. de la Zerda A, Liu Z A, Bodapati S, Teed R, Vaithilingam S, Khuri-Yakub B T, et al. Ultrahigh Sensitivity Carbon Nanotube Agents for Photoacoustic Molecular Imaging in Living Mice. Nano Letters. 2010; 10(6):2168-72.
22. Bouchard L S, Anwar M S, Liu G L, Hann B, Xie Z H, Gray J W, et al. Picomolar sensitivity MRI and photoacoustic imaging of cobalt nanoparticles. P Natl Acad Sci USA. 2009; 106(11):4085-9.
23. Wustholz K L, Henry A I, McMahon J M, Freeman R G, Valley N, Piotti M E, et al. Structure-Activity Relationships in Gold Nanoparticle Dimers and Trimers for Surface-Enhanced Raman Spectroscopy. J Am Chem Soc. 2010; 132(31):10903-10.
24. Yang X M, Stein E W, Ashkenazi S, Wang L H V. Nanoparticles for photoacoustic imaging. Wires Nanomed Nanobi. 2009; 1(4):360-8.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to measurement technique and numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

We claim at least the following:

1. A composition, comprising:
a nanoparticle having a platinum nanostructure and at least one gold nanostructure, wherein the platinum nanostructure and a gold nanostructure are directly attached to one another.

2. The composition of claim 1, wherein the nanoparticle includes one gold nanostructure, wherein the nanoparticle has a dumbbell shape.

3. The composition of claim 2, wherein the gold nanostructure has a diameter of about 4 nm and the platinum nanostructure has a diameter of about 3 nm.

4. The composition of claim 1, wherein the nanoparticle includes two gold nanostructures, wherein each gold nanostructure is independently attached to the platinum nanostructure, wherein the gold nanostructures are not directly attached to one another, wherein the nanoparticle has a dipod shape.

5. The composition of claim 4, wherein each gold nanostructure has a diameter of about 4 to 5 nm and the platinum nanostructure has a diameter of about 4 nm.

6. The composition of claim 1, wherein the nanoparticle includes three gold nanostructures, wherein each gold nanostructure is independently attached to the platinum nanostructure, wherein the gold nanostructures are not directly attached to one another, wherein the nanoparticle has a tripod shape.

7. The composition of claim 6, wherein each gold nanostructure has a diameter of about 6 nm and the platinum nanostructure has a diameter of about 6 nm.

8. The composition of claim 1, wherein the nanoparticle includes four gold nanostructures, wherein each gold nanostructure is independently attached to the platinum nanostructure, wherein the gold nanostructures are not directly attached to one another, wherein the nanoparticle has a tetrapod shape.

9. The composition of claim 8, wherein each gold nanostructure has a diameter of about 6 to 7 nm and the platinum nanostructure has a diameter of about 8 nm.

10. The composition of claim 1, wherein the nanoparticle has a detectable photoacoustic signal and a detectable optical signal.

11. The composition of claim 1, wherein the diameter or the effective diameter is about 5 nm to 40 nm.

12. The composition of claim 1, wherein the diameter or the effective diameter is about 5 nm to 20 nm.

13. The composition of claim 1, wherein the diameter or the effective diameter is about 6 nm to 15 nm.

14. The composition of claim 1, wherein the nanoparticle includes at least two gold nanostructures, wherein each gold nanostructure is independently attached to the platinum nanostructure, wherein the gold nanostructures are not directly attached to one another.

15. The composition of claim 1, wherein the nanoparticle includes at least three gold nanostructures, wherein each gold nanostructure is independently attached to the platinum nanostructure, wherein the gold nanostructures are not directly attached to one another.

16. The composition of claim 1, wherein the nanoparticle has a targeting agent attached to the nanoparticle and wherein the targeting agent is selected from a polypeptide, an antigen, a nucleic acid, a polysaccharide, a sugar, a fatty acid, a steroid, a purine, a pyrimidine, a ligand, an aptamer, a small molecule, or a combination thereof, wherein the targeting agent has an affinity for a condition, disease, or related biological event or other chemical, biochemical, and/or biological events of the condition, disease, or biological event.

17. A method of imaging a tumor, comprising:
exposing a subject to an imaging device, wherein a nanoparticle is introduced to a subject, wherein the nanoparticle enters the tumor, wherein the nanoparticle has a platinum nanostructure and at least one gold nanostructure, wherein the platinum nanostructure and a gold nanostructure are directly attached to one another; and
detecting the nanoparticles, wherein the location of the nanoparticles correlates to the location of the tumor.

18. The method of claim 17, wherein the detection is conducted in vitro or in vivo.

19. The method of claim 17, wherein the imaging device is selected from a photoacoustic device, a Raman imaging device, a PET imaging device, or a combination thereof.

20. The method of claim 17, wherein the nanoparticle includes at least two gold nanostructures, wherein each gold nanostructure is independently attached to the platinum nanostructure, wherein the gold nanostructures are not directly attached to one another.

21. The method of claim 17, wherein the nanoparticle includes at least three gold nanostructures, wherein each gold nanostructure is independently attached to the platinum nanostructure, wherein the gold nanostructures are not directly attached to one another.

22. A method of imaging a disease, comprising:
exposing a subject to an imaging device, wherein a nanoparticle is introduced to a subject, wherein the nanoparticle has a platinum nanostructure and at least one gold nanostructure, wherein the platinum nanostructure and a gold nanostructure are directly attached to one another, wherein the particle becomes disposed adjacent or within the diseased area; and
detecting the nanoparticles, wherein the location of the nanoparticles correlates to the location of the disease.

23. The method of claim 22, wherein the detection is conducted in vitro or in vivo.

24. The method of claim 22, wherein the imaging device is selected from a photoacoustic device, a Raman imaging device, a PET imaging device, or a combination thereof.

25. A method of treating a condition, disease, or related biological event, comprising: administering a pharmaceutically effective amount of a nanoparticle, wherein the nanoparticle has a platinum nanostructure and at least one gold nanostructure, wherein the platinum nanostructure and a gold nanostructure are directly attached to one another, wherein the nanoparticle has a targeting agent attached to the nanoparticle and wherein the targeting agent is selected from a polypeptide, an antigen, a nucleic acid, a polysaccharide, a sugar, a fatty acid, a steroid, a purine, a pyrimidine, a ligand, an aptamer, a small molecule, or a combination thereof, wherein the targeting agent has an affinity for a condition, disease, or related biological event or other chemical, biochemical, and/or biological events of the condition, disease, or biological event.
